(19) United States
(12) Patent Application Publication
(10) Pub. No.: US 2007/0287910 A1
(43) Pub. Date: Dec. 13, 2007

(54) Quick Disconnect and Repositionable Reference Frame for Computer Assisted Surgery

(76) Inventors: Jody Stallings, Palm Harbor, FL (US); Lauralan Terrill-Grisoni, Cordova, TN (US); Kevin Raburn, Memphis, TN (US)

Correspondence Address: CHIEF PATENT COUNSEL;SMITH & NEPHEW, INC., 1450 BROOKS ROAD, MEMPHIS, TN, 38116, US

(21) Appl. No.: 10/594,049

(22) PCT Filed: Apr. 14, 2005
(86) PCT No.: PCT/US05/12566

§ 371 Date: Jun. 20, 2007

Related U.S. Application Data
(60) Provisional application No. 60/563,374, filed on Apr. 15, 2004.

(51) Int. Cl. A61B 19/00

(52) U.S. Cl. 600/426; 600/429; 606/130

(57) ABSTRACT

Devices and processes for use in computer aided or computer navigated surgery include one or more locator and locking mechanisms and mating parts, or, alternatively, one or more adjustable locking structures interposed between an indicium and an item to be used during surgery such as a body part, tool, implant, trial or other structure or component. After the indicia have been registered into the system and surgery begun, it is sometimes desirable to remove and reattach the indicia for better access to the surgical sight, to avoid contamination of the indicia, or to accommodate other surgical needs. Locator and locking mechanisms and mating parts according to various embodiments are designed to be easily removable and accurately reattached so that the indicia can be repositioned relative to the item without the need to reregister the indicia into the system relative to the item. The locator and locking mechanism and mating parts preferably include structure that allows the indicia to be repositioned relative to the item in only one position and orientation, the position and orientation in which it was registered into the system originally. At other times during the surgery, it is desirable to selectively reposition the indicia to conform to anatomical constraints, for better array visibility, or to accommodate other surgical needs. In this situation, the adjustable locking structure allows quick and secure repositioning of the indicia with respect to the underlying item so that the constraints may be accommodated and the surgery may proceed with the assistance of computer guidance. The adjustable locking structure preferably includes structure to allow selective repositioning when desired but requires the adjustable locking structure to remain rigidly fixed when movement is not desired.

### CROSS-REFERENCE TO RELATED APPLICATION

This application claims the benefit of U.S. Provisional Application Ser. No. 60/563,374 entitled “Quick Disconnect and Repositionable Reference Frame for Computer Assisted Surgery” filed on Apr. 15, 2004, the contents of which are incorporated herein by reference.

### FIELD OF THE INVENTION

The present invention relates to reference frame attachments for use in computer aided surgical navigation, and methods for their use. More specifically, the invention relates to reference frame attachments comprising indicia, either passive such as fiducials, or active such as transponders, which can be sensed by a computer aided surgical navigation system. Such reference frames are coupled to body parts, tools and surgical components so that the system may track and display, as desired, their position and orientation during surgery. Reference frame attachments according to certain embodiments of the present invention are designed to be detached at desired times during the surgery and accurately reattached into correct position with respect to their original registration into the surgical navigation system. Alternatively, the reference frame attachments may be conveniently repositioned with respect to the body part, tool or component to which they are coupled in order to comply with anatomical constraints, improve visibility of the indicia, or for other purposes.

### BACKGROUND

A major concern during surgical procedures, as well as other medical operations, is conducting the procedures with as much precision as possible. For example, in orthopedic procedures, less than optimum alignment of implanted prosthetic components may cause undesired wear and revision, which may eventually lead to the failure of the implanted prosthesis. Other general surgical procedures also require precision in their execution.

With orthopedic procedures, for example, previous practices have left room for precision alignment of prosthetic components. For example, in a total knee arthroplasty, previous instrument design for resection of bone limited the alignment of the femoral and tibial resections to average value for varus/valgus, flexion/extension and external/internal rotation. Additionally, surgeons conventionally often use visual landmarks or “rules of thumb” for alignment which can be misleading due to anatomical variability. Intramedullary referencing instruments can also violate the femoral and tibial canal. This intrusion can increase the risk of fat embolism and unnecessary blood loss in the patient, among other things.

Devices and processes according to various embodiments of the present invention are applicable not only for knee, hip and shoulder repair, reconstruction or replacement surgery, but also repair, reconstruction or replacement surgery in connection with any other joint of the body as well as any other surgical or other operation where it is useful to track position and orientation of body parts, non-body components and/or virtual references such as rotational axes, and to display and output data regarding positioning and orientation of them relative to each other for use in navigation and performance of the operation.

Several manufacturers currently produce image-guided surgical navigation systems that are used to assist in performing surgical procedures with greater precision. The TREON™ and iON™ systems with FLUORONAV™ software manufactured by Medtronic Surgical Navigation Technologies, Inc. are examples of such systems. The BrainLAB VECTORVISION™ system is another example of such a surgical navigation system. Systems and methods for accomplishing image-guided surgery are also disclosed in U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; U.S. Ser. No. 10/229,372, filed Aug. 27, 2002 and entitled “Image Computer Assisted Knee Arthroplasty”; U.S. Ser. No. 10/084,278 filed Feb. 27, 2002 and entitled “Total Knee Arthroplasty Systems and Processes,” which claims priority to provisional application entitled “Surgical Navigation Systems and Processes,” Ser. No. 60/355,899, filed Feb. 11, 2002; U.S. Ser. No. 10/084,278 filed Feb. 27, 2002 and entitled “Surgical Navigation Systems and Processes for Unicompartmental Knee Arthroplasty,” which claims priority to provisional application entitled “Surgical Navigation Systems and Processes,” Ser. No. 60/355,899, filed Feb. 11, 2002; U.S. Ser. No. 10/084291 entitled Surgical Navigation Systems and Processes for High Tibial Osteotomy,” which claims priority to provisional application entitled “Surgical Navigation Systems and Processes,” Ser. No. 60/355,899, filed Feb. 11, 2002; provisional application entitled “Image-guided Navigated Precisions Reamers,” Ser. No. 60/474,178, filed May 29, 2003, and U.S. Ser. No. 10/689,103, filed Oct. 20, 2003, entitled “Magnetic Modular Fiducials,” the entire contents of each of which are incorporated herein by reference as are all documents incorporated by reference therein.

These systems and processes use position and/or orientation tracking sensors such as infrared sensors acting stereoscopically or other sensors acting in conjunction with reference structures or reference transmitters to track positions of body parts, surgery-related items such as implements, instrumentation, trial prosthetics, prosthetic components, and virtual constructs or references such as rotational axes which have been calculated and stored based on designation of bone landmarks. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated reference structures such as fiducials, reference transmitters, or based on stored position and/or orientation information. The processing functionality correlates this position and orientation information for each object with stored information, such as a computerized fluoroscopic imaged file, a wire frame or other data file for rendering a representation of an instrument component, trial prosthesis or actual prosthesis, or a computer generated file relating to a rotational axis or other virtual construct or reference. The processing functionality then displays position and orientation of these objects on a screen or monitor, or otherwise. Thus, systems or processes, by sensing the position of indicia such as passive fiducials or active transmitters or transponders which may be located on reference frames, can display or otherwise output useful data relating to predicted or actual position and orientation of body parts, surgically related items, implants, and virtual constructs for use in navigation, assessment, and otherwise performing surgery or other operations.

Indicia according to certain aspects of the invention may emit or reflect infrared light that is then detected by an infrared camera. They may be sensed actively or passively by infrared, visual, sound, magnetic, electromagnetic, x-ray or any other desired technique. An active indicium emits energy, and a passive indicium merely reflects energy. A reference frame may have at least three, but usually four, indicia to determine the position and orientation of the associated instrument, implant component or other object to which the reference frame is attached. In this sense, some or all of the indicia could be part of the same small structure; for example, the indicia could be three nubs, indicating spikes, small spheres, or other irregularities.

In addition to reference frames with fixed indicia, modular indicia, which may be positioned independent of each other, may be used to reference points in the coordinate system. Modular indicia may include reflective elements which may be tracked by one, two, sometimes more sensors whose output may be processed by associated processing functionality to geometrically calculate the position and orientation of the item to which the modular indicia are attached. Like fixed reference frame structures, modular reference frames and the sensors need not be confined to the infrared spectrum—any electromagnetic, electrostatic, light, sound, radio frequency or other desired technique may be used. Similarly, modular indicia may “actively” transmit reference information to a tracking system, as opposed to “passively” reflecting infrared or other forms of energy.

Some image-guided surgical navigation systems allow reference frame structures with indicia to be detected at the same time the fluoroscopy imaging is occurring. This allows the position and orientation of the reference structure to be coordinated with the fluoroscope imaging. Then, after processing position and orientation data, the reference structures may be used to track the position and orientation of anatomical features that were recorded fluoroscopically. Computer-generated images of instruments, components, or other structures that are fitted with reference frame structures may be superimposed on the fluoroscopic images. The instruments, trial, implant or other structure or geometry can be displayed as 3-D models, outline models, or bone-implant interface surfaces.

Some reference frame structures also feature indicia which are arranged in particular patterns, so that the computer aided surgical navigation system can discern one reference frame from another, and thus various body parts, tools and other items from each other as they are being tracked.

The precise spatial relationship of individual indicia with respect to each other and the associated anatomy or instrument forms the basis of how an indicia-based system calculates the position and orientation of the associated items. Consequently, once the spatial relationship of the fiducials or reference transmitter with respect to each other and with respect to the associated body part or item to be tracked has been registered in the computer aided surgical navigation system, subsequent changes in the position and/or orientation of the indicia relative to each other or to the body part or item will likely cause the system to erroneously calculate the position and orientation of such parts or items. Even minor changes in orientation and/or position of the reference frames or indicia mounted on them may lead to dramatic differences in how the system detects the orientation and/or location of the associated anatomy or instruments. Such changes may require the system to be recalibrated, requiring additional fluoroscopy or other imaging to be obtained, increasing the time and the expense of the procedure. Failure to recalibrate the system may lead to imprecision in the execution of the desired surgical procedure.

In addition to requiring a high level of precision, surgical procedures often present challenging anatomical constraints on the size and shape of equipment that may be used during the procedure. While referencing frames are critical for certain aspects of the procedure, they can also be prohibitively cumbersome or disruptive due to their size, shape, or orientation.

During the surgical procedure, there are times when it is desirable to temporarily remove the referencing structure or one or more indicia in order to accommodate anatomical constrains, improve access to a surgical site, or for other considerations. Currently, there is no convenient way to temporarily detach the referencing structure or fiducials and reattach them in their correct position. If the reference structures and/or fiducials are not replaced in position accurately, they will provide inaccurate information about the location, position, and orientation of the body parts, non-body components and other reference points previously placed in the coordinate system and the accuracy and safety of the surgical procedure may be jeopardized. And, as discussed above, even the slightest change can have dramatic results.

Similarly, there is currently no convenient way to reposition the reference frame or indicia with respect to a surgical tool or component in order to permit continued use of that item while still adhering to the particular anatomical constraints of the surgical site.

As a result, the surgeon must either leave the reference frame and fiducials in place, which may be cumbersome or interfere with the procedure, or he must remove the reference frame structure, which prevents utilizing the computer assisted guidance. After removing the reference frame, and with no precise way to reattach the referencing structure, if the surgeon wants to continue with the image guided surgery, he must reregister each instrument that will be used in the procedure and each reference structure and fiducial that is on the patient or otherwise in the coordinate system. This process lengthens the time necessary to complete the surgical procedure and can result in unnecessary complications resulting from the additional length of time the patient is in surgery.

Adding to this concern is the tendency of some surgeons to not take the time necessary to recalibrate the entire system when a reference structure or indicium is dislocated by being inadvertently bumped. When this occurs, the virtual image created by the imaging system is not a true reflection of the actual position, orientation and relationship of the body parts, non-body components and other reference points. Proceeding with surgical procedures with a coordinate system under these conditions can lead to obvious problems.

Another need for a removable reference frame structure is created by the fact that the risk of contamination of the indicia is proportional to the length of time the indicia are in use. Thus the risk of contamination is reduced if the indicia are only attached at certain required times during the surgical procedure instead of remaining attached throughout the entire operation. When the entire operation is lengthy, but the computer assisted guidance is only required for certain critical aspects of the surgery, leaving the referencing frame or indicia attached throughout the entire procedure unnecessarily increases the risk of indicia contamination.

Certain aspects of a surgical procedure may present anatomical constraints or other factors inhibiting the use of the referencing structure but not permit removal of the referencing structure completely. This would occur, for example, in situations where the surgeon needed to maintain the computer guidance of a particular instrument, but where the position of the referencing structure as originally registered on the instrument does not allow for proper use of the instrument during the surgery. This could happen because the protruding referencing structure was blocked by a body part, or for any number of other reasons.

Additionally, certain orientations of the referencing frame may result in a positioning of the indicia such that there is a low array visibility. This inhibits the computer's ability to properly map the position of the tracked body parts or items. When this occurs, it may be necessary to reposition the reference frame which may necessitate reorienting it with respect to the underlying surgical item. This allows the underlying surgical item to be oriented as required by the procedure while also enabling the reference frame structure to be repositioned for increased array visibility.

Because of the problems above, among others, it is desirable for a referencing frame or indicia attached thereto to remain fixed in place when movement is not desired, but to be easily removable or repositionable, when necessary, so that surgical constraints may be accommodated. Existing fixed reference frames and breakaway reference frames and fiducials do not address these problems.

### SUMMARY

Various aspects and embodiments of the present invention include reference frame attachments with portions that can readily disconnect from a base secured to a body part, surgical tool or item or other component. Such attachments can be precisely repositioned after removal, and can remain securely attached when removal is not desired. Alternatively, aspects of some embodiments of the present invention allow for easy and accurate deliberate repositioning of the reference frame with respect to the body part, surgical tool, or other item when required by anatomical constraints, array visibility or other factors.

According to one aspect of the present invention, a reference frame attachment includes a connecting portion with an interface designed to complement the receiving portion of a base secured to a body part or item. The attachment device creates a stable connection in a fixed position with the base, but it may easily be separated from and reattached to the base in the same fixed position without resulting in a change of location of the base within the coordinate system or a change of location of the body part or instrument to which the device is attached. The attachment can then be easily replaced without having to recalibrate the entire system or disturb the underlying body part or instrument.

According to another aspect, a frame attachment includes a connecting portion with an interface which is designed to complement a receiving portion of a base. The attachment device creates a stable connection with the base through the use of a registering and securing mechanism. Such registering and securing mechanism may comprise a magnetic attraction, adhesive, hook and pile connectors, ball and plunger, tongue and groove, key and hole, or any other material or force which creates a suitable bond between the attachment device and base and which would require the device to be properly attached only in the desired position. As such, the attachment device can be easily removed and replaced without the need to recalibrate the entire system or disturb the underlying body part or instrument.

According to another aspect, a frame attachment includes an adjustable securing mechanism designed to connect a referencing frame to an underlying item. The adjustable securing mechanism creates a stable connection between the referencing frame and an underlying part in a variety of selectable positions. The frame attachment may be easily and securely repositioned to accommodate anatomical constraints, for improved array visibility, or to meet other surgical needs.

According to other aspects of the present invention, the attachment device comprises indicia such as passive fiducials or active reference transmitters or transponders, as well as any other desired reference devices.

According to other aspects of the present invention, reference structures and modular fiducials exhibit modularity such that they may be moved within a coordinate system without the disruption of the base secured within the coordinate system.

### BRIEF DESCRIPTION OF THE DRAWINGS

FIG. 1 shows a portion of a reference frame attachment according to one embodiment of the invention, this portion including a registering and securing mechanism;

FIG. 2 shows another portion of a reference frame attachment, this portion adapted to register with and secure to the portion of the reference frame shown in FIG. 1;

FIG. 3 shows the portion of the reference frame attachment shown in FIG. 1 and the portion of the reference frame attachment shown in FIG. 2 registered and secured together;

FIG. 4 shows a portion of a reference frame attachment according to another embodiment of the invention, this portion including an alternative registering and securing mechanism;

FIG. 5 shows another portion of a reference frame configured to register with and securely attach to the portion of the reference frame of FIG. 4;

FIG. 6 shows the portion of the reference frame of FIG. 4 positioned to register and secure the portion of the reference frame show in FIG. 5;

FIG. 7 is a side view of a reference frame attachment according to another embodiment of the invention, this portion including an adjustable securing mechanism; and

FIG. 8 is a front view of the adjustable securing mechanism of FIG. 7.

### DETAILED DESCRIPTION OF THE INVENTION

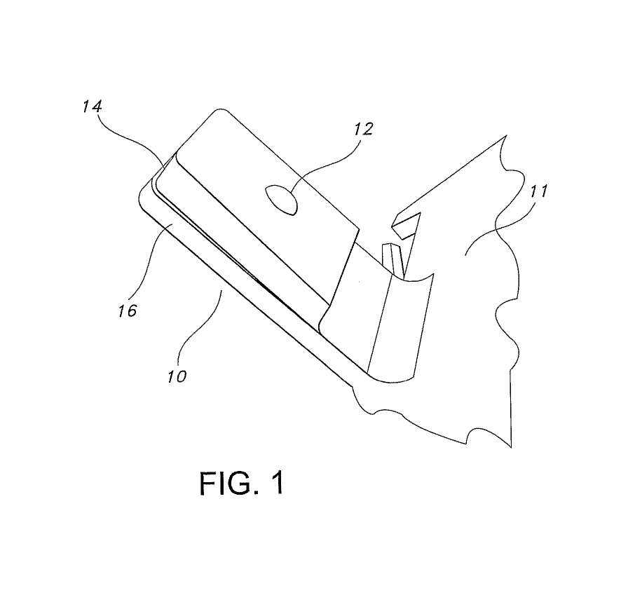

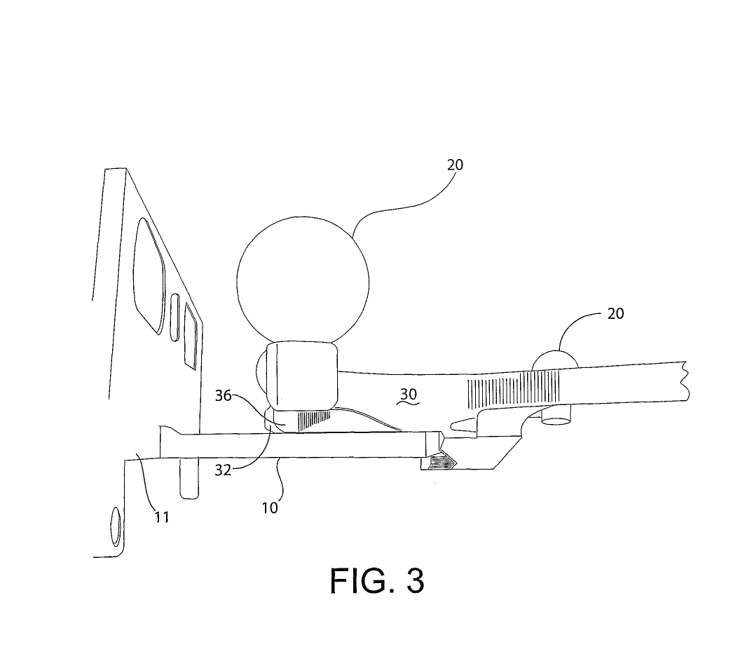

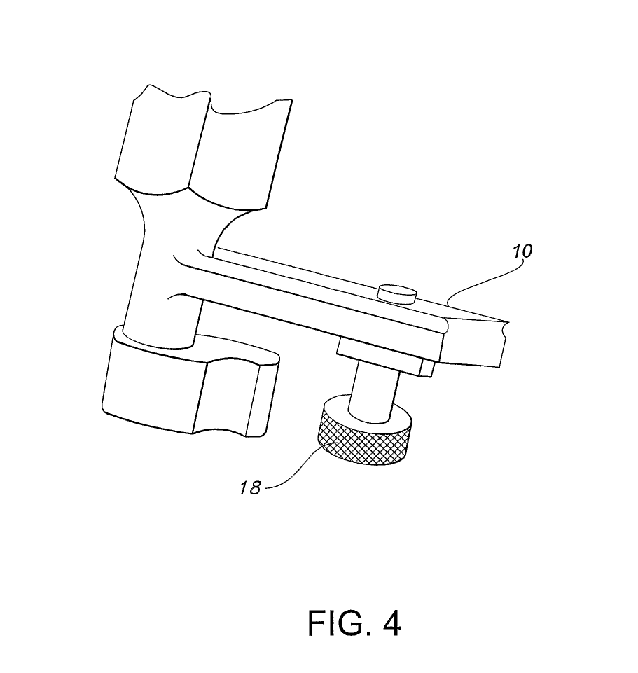

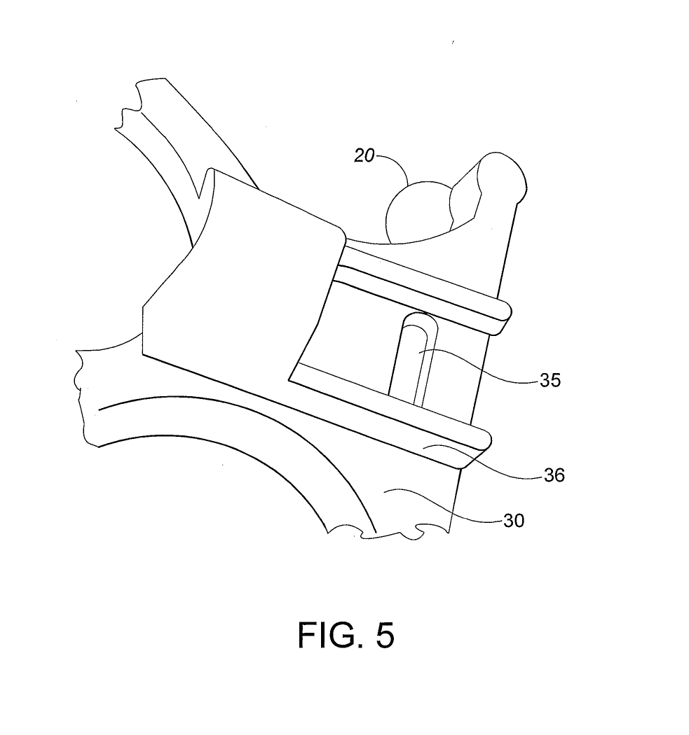

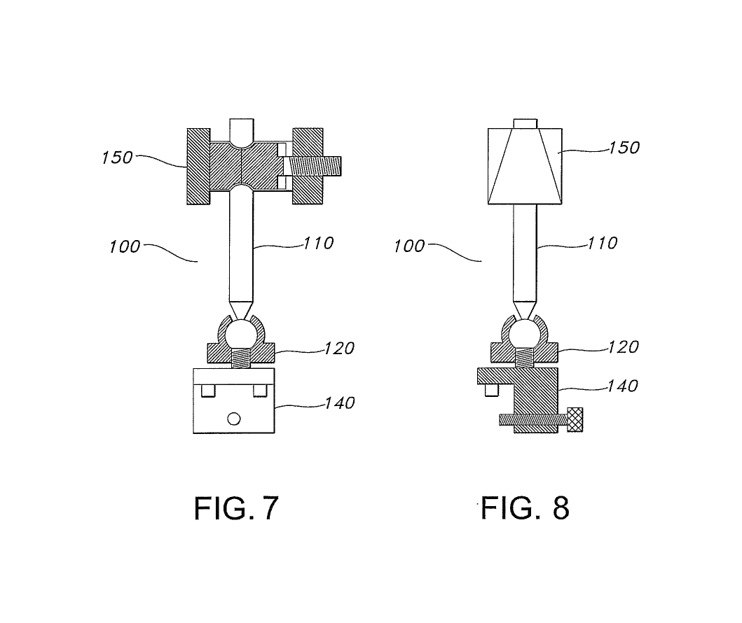

Various embodiments of the present invention may be constructed. FIGS. 1-3 illustrate one example embodiment of the present invention. FIGS. 4-6 illustrate an alternative embodiment of the present invention, and FIGS. 7-8 illustrate another alternative embodiment of the present invention. The present invention may be embodied in other forms as well.

The present invention, in one aspect, comprises a computer aided surgery system, the computer aided surgery system comprising a sensor adapted to sense position of one or more indicia 20, a computer functionality adapted to receive information from the sensor about position of the indicia 20 and generate information corresponding to position and orientation of an item to which the indicia 20 are attached.

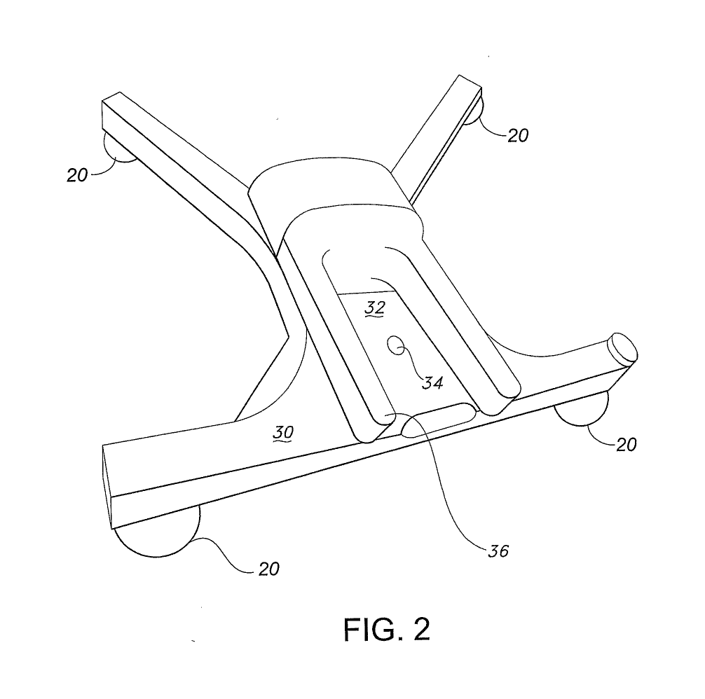

According to some embodiments, the present invention further comprises a reference frame, the reference frame having a first portion 10, illustrated in an example embodiment in FIG. 1, and a second portion 30 illustrated in an example embodiment in FIG. 2. The reference frame further comprises a registering and securing mechanism.

According to some embodiments, the registering and securing mechanism may be used to register the first portion 10 of the reference frame with respect to the second portion 30 of the reference frame so that a desired orientation of the first portion 10 of the reference frame with respect to the second portion 30 of the reference frame is achieved. Examples of this aspect of the present invention are illustrated in FIGS. 1-6. The registering and securing mechanism, according to some aspects of some embodiments, securely attaches the first portion 10 and second portion 30 of the reference frame to each other so that there is no undesired movement of the first portion 10 of the reference frame with respect to the second portion 30 of the reference frame.

According to some embodiments, the registering and securing mechanism securely attaches the first portion 10 and the second portion 30 of the reference frame in the desired orientation achieved by use of the registering and securing mechanism. In use, the indicia 20 and the first and second portions of the reference frame may be attached to an item used in surgery. According to the example embodiment shown in FIG. 1, an attachment portion 11 can be used to facilitate attachment to an item used in surgery. The item used in surgery may be a body part, a surgical instrument, a prosthetic implant, a surgical rod or support device, or any other item. According to some aspects of some embodiments, the indicia 20 may attach to the first portion 10 of the reference frame and the second portion 30 of the reference frame may attach to the item used in surgery. During use, a surgeon may register and secure the first portion 10 of the reference frame with respect to the second portion 30 of the reference frame so that the indicia 20 are attached by way of the reference frame to the item used in surgery.

After registering the indicia 20 with the computer aided surgery system, the surgeon may then detach the first portion 10 of the reference frame and the indicia 20 from the second portion 30 of the reference frame and the item used in surgery in order to facilitate the surgical procedure by increasing access to the surgical site, reducing the risk of indicium contamination, or otherwise aiding the procedure.

After detaching the first portion 10 of the reference frame from the second portion 30, certain embodiments of the present invention allow the surgeon to register and resecure the first portion 10 of the reference frame to the second portion 30 so that the position and orientation of the indicia 20 with respect to the item used in surgery is unchanged from the position and orientation of the indicia 20 with respect to the item used in surgery when originally registered with the computer aided surgery system. This may be accomplished through the use of a registering mechanism which is configured to allow the first portion 10 of the reference frame to connect to the second portion 30 of the reference frame in only a desired position and orientation, and a securing mechanism which selectively prohibits movement of the first portion 10 of the reference frame with respect to the second portion when the desired position and orientation is achieved. The registering mechanism and the securing mechanism are preferably, but not necessarily, part of the same structure comprising a registering and securing mechanism.

According to certain aspects of the present invention the registering and securing mechanism may function so that the first and second portions of the reference frame engage each other in a sliding relationship. According to some aspects, a mating section 14, illustrated in FIG. 1, of the first portion 10 of the reference frame may be shaped to join with a mating section 32, illustrated in FIG. 2, of the second portion 30 so that the first portion 10 is allowed to move only along one degree of translational freedom with respect to the second portion 30, all other degrees of freedom being restricted by structure of the reference frame. According to some embodiments the registering and securing mechanism may then determine at what point along the one degree of translational freedom the first portion 10 becomes securely fixed in all degrees of freedom so that all undesired movement of the first portion 10 of the reference frame with respect to the second portion 30 is enabled.

Referring to FIGS. 1-3, which illustrate various aspects of an example embodiment, the registering and securing mechanism may include a ball plunger 12 on the first portion 10 of the reference frame and a receiving recess 34, shown in FIG. 2, on the second portion 30 of reference frame. In other embodiments, other registering and securing mechanisms may be employed. For example, according to one embodiment, a d-shaped male pin is configured to be received by a d-shaped female receptor. The d-shaped male pin may employ protrusions perpendicular to a shaft of the pin to engage openings in the female receptor. The male/female assembly can further be spring loaded to allow a positive lock once a desired alignment is achieved. According to aspects of embodiments employing a ball plunger 12, shown in FIG. 1, and the receiving recess 34, when the first portion 10 is registered with respect to the second portion 30 in the desired orientation, the ball plunger 12 is positioned to engage the receiving recess 34 so that it prevents further movement of the first portion 10 with respect to the second portion 30 along the degree of translational freedom allowed by the sliding action of the first and second portions 10 and 30.

According to other aspects, the present invention may be configured to allow unsecuring the first portion 10 of the reference frame from the second portion 30 of the reference frame without disturbing the item used in surgery and with minimal force so that there is no risk of a surgeon's hand slipping or bumping another item.

This can be achieved in various ways. In the embodiment illustrated in FIGS. 4-6, by a registering and securing mechanism is configured so that the two portions of the reference frame may be unsecured by applying a minimal force to the registering and securing mechanism together with a force to another part of the registering and securing mechanism or area immediately around the registering and securing mechanism such that there is little or no net resulting force on the reference frame. According to some aspects of some embodiments, this is achieved by applying an upward force to the retractable plunger 18, illustrated in FIG. 4, together with a downward force on a portion of the reference frame immediately below the retractable plunger 18 as would happen if one's thumb was on the reference frame while one's index finger and middle finger were used to lift the retractable plunger 18.

Similarly, the registering and securing mechanism, according to some aspects of the invention may be unsecured by applying a force in one direction on a first side of thumb screw while also applying an opposing force in an opposite direction on a second side of a thumb screw such as would happen when one was twisting a thumb screw.

Referring again to FIGS. 1-3, in other aspects of some embodiments movement in degrees of freedom other then the single translational degree along the desired sliding direction is enabled by shaping of the mating section 14, shown in FIG. 1, of the first portion 10 and second portion 30, shown in FIG. 2, of the reference frame. This shaping may include, for example, a groove and flange 36, illustrated in FIG. 2, along the second portion 30 that receives a corresponding member 16 of the first portion 10 configured to mate with the groove and flange 36 as illustrated in example FIG. 1.

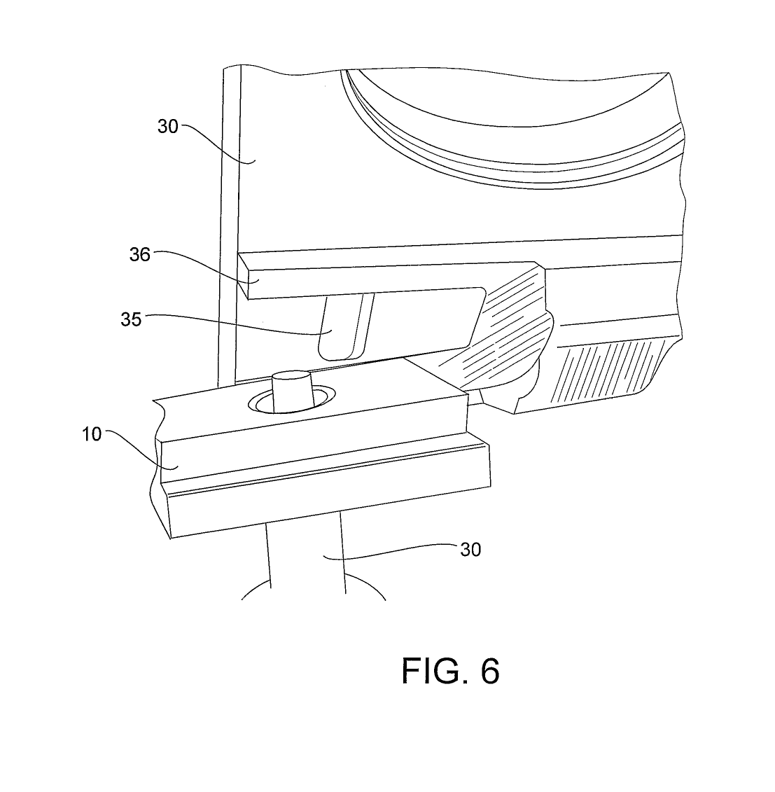

According to other aspects of some embodiments, a retractable plunger 18, illustrated in FIG. 4, may be used in place of a ball plunger 12, as shown in FIG. 1, as a registering and securing mechanism for registering the first portion 10 with respect to the second portion 30 in a desired position and for securing the first portion 10 of the reference frame with respect to the second portion 30 of the reference frame in the desired position achieved by the registering and securing mechanism. Referring now to FIG. 6, which illustrates one aspect of an embodiment employing a retractable plunger 18 comprising a receiving groove 35.

Other aspects of some embodiments could include as part of the registering and securing mechanism a magnetic mechanism, either permanent or induced by an electric current, a snap mechanism, a spring catch mechanism, a latching mechanism, a buckle mechanism, a male/female pin and receptor, or any combination of these or a similar type of binding or fastening mechanisms that secure the first portion 10 of the reference frame with respect to the second portion 30 of the reference frame. This list is not exhaustive, other embodiments of registering and securing mechanisms could be apparent to one skilled in the art and are within the scope of the present invention.

According to other aspects of some embodiments, the registering and securing mechanism could include a tongue and groove mechanism, a dovetail mechanism, a key and hole mechanism, a flange and tab mechanism, a rim and collar mechanism, or any similar mechanism for corresponding the first portion 10 of the reference frame relative to the second, or any combination of these mechanisms. This list is not exhaustive, other embodiments of registering and securing mechanisms could be apparent to one skilled in the art and are within the scope of the present invention.

Referring now to the embodiment illustrated in FIGS. 7-8: according to certain aspects of the embodiment illustrated in FIGS. 7-8, structures according to such aspects of the present invention may comprise a reference frame, the reference frame comprising a first portion 150 and a second portion 140. The first portion of the reference frame 150, according to certain embodiments, may be joined to the second portion 140 of the reference frame by an adjustable securing mechanism 100. The adjustable securing mechanism 100, according to some aspects of some embodiments, allows the first portion 150 selectively to be either fixed or moveable relative to the second portion 140 of the reference frame.

In use, a surgeon may selectively configure the adjustable securing structure to allow the first portion 150 of the reference frame to move relative to the second portion 140 and then move the first portion 150 of the reference frame into a desired location and orientation relative to the second portion 140. The surgeon may then selectively configure the adjustable securing mechanism to secure the first portion 150 of the reference frame in a fixed position and orientation relative to the second portion 140 of the reference frame.

According to some aspects of some embodiments, the first portion 150 of the reference frame, as illustrated in FIG. 7, may be attached to one or more indicia. The second portion 140 of the reference frame may, according to some aspects of some embodiments, be attached to an item used in surgery. Accordingly, when the first portion 150 is attached to one or more indicia and the second portion 140 is attached to an underlying item, movement or orientation of the first portion 150 relative to the second portion 140, moves or orients the indicia relative to the item used in surgery.

During a procedure, the surgeon may selectively configure an adjustable locking structure to allow the indicia to be positioned relative to the item used in surgery and then selectively configure the adjustable locking structure to secure the first portion 150 of the reference frame, illustrated in FIGS. 7-8, with respect to the second portion 140, illustrated in FIGS. 7-8, and thereby secure the position and orientation of the indicia relative to the item used in surgery.

Once the indicia are secured relative to the item used in surgery, the indicia may be registered with the computer aided surgery system. After registering the indicia, if the surgeon desires to move the indicia or reposition the indicia relative to the underlying item used in surgery, but still proceed with the assistance of the computer aided surgery system, the surgeon may selectively configure the adjustable locking mechanism to allow the indicia to be repositioned or re-oriented with respect to the item used in surgery. Once a desired new position and orientation is achieved, the surgeon may then selectively configure the adjustable locking structure to secure the first portion 150 of reference frame with respect to the second portion 140 and thereby secure the position and orientation of the indicia relative to the item used in surgery in the desired new position. Once the desired new position is secured, the surgeon may re-register the indicia with the computer aided surgery system and proceed with the surgical procedure.

In the example embodiment illustrated in FIGS. 7-8, the adjustable locking mechanism includes an adjustable rod 110 and a thumb screw 120. The adjustable rod 110 and thumb screw 120 may be configured so that loosening the thumb screw 120 releases tension on the adjustable rod 110 thereby enabling it to rotate relative to the thumb screw 120. According to some aspects of the embodiments, the adjustable rod 110 may be attached to the first portion 150 of the reference frame and the thumb screw 120 may be attached to the second portion 140 of the reference frame so that when the thumb screw 120 is loosened and the adjustable rod 110 is allowed to rotate relative to the thumb screw 120, the first portion 150 of the reference frame is thereby allowed to rotate relative to the second portion 140 of the reference frame.

According to other aspects of some embodiments, the adjustable locking structure may include a hinge, a swivel, a flexible rod, or any other articulating mechanism or combination of the above that permits the first portion 150 of the reference frame to move or rotate with respect to the second portion 140. This list is not exhaustive, other embodiments of adjustable securing mechanisms could be apparent to one skilled in the art and are within the scope of the present invention.

1. A system for computer aided surgery navigation which includes a sensor adapted to sense position of a plurality of indicia attached by a reference frame to an item used in surgery and a computer functionality adapted to receive information from the sensor about position of the indicia and generate information corresponding to position and orientation of the item to which the indicia are attached:
wherein the indicia are attached to the item using at least one registering and securing mechanism such that the indicia may attach only in a determined position and so that the indicia may be removed from the item and reattached without incorrect registration relative to the item; and wherein the registering and securing mechanism features a structure which allows the indicia to be selectively attached and detached from the item.

2. A system according to claim 1 wherein at least one of the indicia includes a reflective surface adapted to be sensed by an infrared sensor device or a transponder that emits energy when interrogated.

3. A system according to claim 1 in which the registering and securing mechanism comprises a separate registering mechanism and a separate securing mechanism.

4. A system accordingly to claim 1 in which the registering and securing mechanism comprises at least one of a ball plunger, a retractable plunger, a male pin and female receptor, or a magnetic device.

5. A device for use in a computer aided surgical navigation system including a sensor adapted to sense position of a plurality of indicia attached by a reference frame to an item used in surgery, and computer functionality adapted to receive information from the sensor about position of the indicia and generate information corresponding to position and orientation of the item to which the indicia are attached comprising:
a reference frame to which the indicia may be attached, the reference frame adapted to be connected to the item;
a registering and securing mechanism interposed between at least one indicium and the item;
wherein the indicia may only attach in a determined position so that they may be removed from the item and reattached without incorrect registration of the indicia relative to the item; and
wherein the registering and securing mechanism includes a structure which allows the indicia to be selectively attached and detached from the item.

6. A device according to claim 5 wherein at least one of the indicia includes a reflective surface adapted to be sensed by an infrared sensor device or a transponder that emits energy when interrogated.

7. A device according to claim 5 in which the registering and securing mechanism comprises a separate registering mechanism and a separate securing mechanism.

8. A device according to claim 5 in which the registering and securing mechanism comprises at least one of a ball plunger, a retractable plunger, a male pin and female receptor, or a magnetic device.

9. A process for conducting a computer aided surgery including providing a computer aided surgery system including a sensor adapted to sense position of a plurality of indicia attached by a reference frame to an item used in surgery and computer functionality adapted to receive information from the sensor about position of the indicia and generate information corresponding to position and orientation of the item to which the indicia are attached:
wherein at least one of the indicia is attached to the item using a registering and securing mechanism such that the indicium may only attach in a determined position so that it may be removed from the item and reattached without incorrect registration of the indicium relative to the item;
wherein the registering and securing mechanism features structure which allows the indicium to be selectively attached and detached from the item;
wherein the indicia are registered into the system;
wherein the item is navigated during surgery using the image rendered by the rendering functionality;
wherein at least one indicium is detached from the item;
wherein the indicium is repositioned into correct position and orientation relative to the item; and
wherein the item continues to be navigated during surgery without the need to reregister the indicium into the system.

10. A computer aided surgery navigation system including a sensor adapted to sense position of a plurality of indicia attached by a reference frame to an item used in surgery and computer functionality adapted to receive information from the sensor about position of the indicia and generate information corresponding to position and orientation of the item to which the indicia are attached:
wherein at least one of the indicia is attached to the item using an adjustable securing mechanism adapted to allow a variety of securely fixed orientations of the indicia relative to the item; and
wherein the adjustable securing mechanism features structure which allows the indicium to be selectively repositioned and resecured relative to the item.

11. A system according to claim 10 in which at least one of the indicia includes a fiducial or an active device.

12. A system according to claim 10 in which the adjustable securing mechanism includes an adjustable rod with a base thumb screw for securing the adjustable rod.

13. A device for use in a computer aided surgical navigation system, the system including a sensor adapted to sense position of a plurality of indicia attached by a reference frame to an item used in surgery; computer functionality adapted to receive information from the sensor about position of the indicia and generate information corresponding to position and orientation of the item to which the indicia are attached, including:
a reference frame adapted to be connected to the item;
at least one indicium connected to the reference frame; and
an adjustable securing mechanism interposed between at least one indicium and the item, wherein the adjustable securing mechanism is configured such that the indicia may attach in a variety of rigidly fixed orientations relative to the item, and wherein the adjustable securing mechanism features structure which allows the indicium to be selectively repositioned and resecured relative to the item.

14. A device according to claim 13 in which at least one of the indicia includes a fiducial or an active device.

15. A device according to claim 13 in which the adjustable securing mechanism includes an adjustable rod with a base thumb screw for securing the adjustable rod.

16. A process for conducting computer aided surgery including providing a computer aided surgery system including a sensor adapted to sense position of a plurality of indicia attached by a reference frame to an item used in surgery, and a computer functionality adapted to receive information from the sensor about position of the indicia and generate information corresponding to position and orientation of the item to which the indicia are attached:
wherein the indicia are attached to the item using an adjustable securing mechanism adapted to allow a variety of rigidly fixed orientations of the indicia relative to the item;
wherein the adjustable securing mechanism features structure which allows the indicia to be selectively repositioned and resecured relative to the item;
wherein the indicia are registered into the system;
wherein the item is navigated during surgery using the image rendered by the rendering functionality;
wherein at least one indicium needs to be repositioned due to an anatomical constraint, reduced array visibility, or other surgical need;
wherein the indicia are selectively repositioned relative to the item;
wherein the indicia are reregistered; and
wherein the item continues to be navigated during surgery without the need to remove the indicia from the underlying item.
